(19) United States
(12) Patent Application Publication
(10) Pub. No.: US 2008/0119479 A1
(43) Pub. Date: May 22, 2008

(54) Combination Comprising Zd6474 And Imatinib

(76) Inventor: Stephen Robert Wedge, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 11/663,913

(22) PCT Filed: Sep. 23, 2005
(86) PCT No.: PCT/GB05/03673

§ 371 Date: Mar. 27, 2007

(30) Foreign Application Priority Data
Apr. 1, 2004 (GB) 0506726.9
Sep. 27, 2004 (GB) 0421438.3

(51) Int. Cl. A61K 31/496; A61P 35/02; A61P 35/00

(52) U.S. Cl. 514/252.18

(57) ABSTRACT

The present invention relates to a method for the production of an antiangiogenic and/or vascular permeability reducing effect in a warm-blooded animal such as a human which is optionally being treated with ionising radiation, particularly a method for the treatment of a cancer, particularly a cancer involving a solid tumour or a leukaemia, which comprises the administration of ZD6474 in combination with imatinib; to a pharmaceutical composition comprising ZD6474 and imatinib; to a combination product comprising ZD6474 and imatinib for use in a method of treatment of a human or animal body by therapy; to a kit comprising ZD6474 and imatinib; to the use of ZD6474 and imatinib in the manufacture of a medicament for use in the production of an antiangiogenic and/or vascular permeability reducing effect in a warm-blooded animal such as a human which is optionally being treated with ionising radiation.

The present invention relates to a method for the production of an antiangiogenic and/or vascular permeability reducing effect in a warm-blooded animal such as a human which is optionally being treated with ionising radiation, particularly a method for the treatment of a cancer, particularly a cancer involving a solid tumour or a leukaemia, which comprises the administration of ZD6474 in combination with imatinib; to a pharmaceutical composition comprising ZD6474 and imatinib; to a combination product comprising ZD6474 and imatinib for use in a method of treatment of a human or animal body by therapy; to a kit comprising ZD6474 and imatinib; to the use of ZD6474 and imatinib in the manufacture of a medicament for use in the production of an antiangiogenic and/or vascular permeability reducing effect in a warm-blooded animal such as a human which is optionally being treated with ionising radiation.

Normal angiogenesis plays an important role in a variety of processes including embryonic development, wound healing and several components of female reproductive function. Undesirable or pathological angiogenesis has been associated with disease states including diabetic retinopathy, psoriasis, cancer, rheumatoid arthritis, atheroma, Kaposi's sarcoma and haemangioma (Fan et al, 1995, Trends Pharmacol. Sci. 16: 57-66; Folkman, 1995, Nature Medicine 1: 27-31). Alteration of vascular permeability is thought to play a role in both normal and pathological physiological processes (Cullinan-Bove et al, 1993, Endocrinology 133: 829-837; Senger et al, 1993, Cancer and Metastasis Reviews, 12: 303-324). Several polypeptides with in vitro endothelial cell growth promoting activity have been identified including, acidic and basic fibroblast growth factors (aFGF & bFGF) and vascular endothelial growth factor (VEGF). By virtue of the restricted expression of its receptors, the growth factor activity of VEGF, in contrast to that of the FGFs, is relatively specific towards endothelial cells. Recent evidence indicates that VEGF is an important stimulator of both normal and pathological angiogenesis (Jakeman et al, 1993, Endocrinology, 133: 848-859; Kolch et al, 1995, Breast Cancer Research and Treatment, 36:139-155) and vascular permeability (Connolly et al, 1989, J. Biol. Chem. 264: 20017-20024). Antagonism of VEGF action by sequestration of VEGF with antibody can result in inhibition of tumour growth (Kim et al, 1993, Nature 362: 841-844).

Receptor tyrosine kinases (RTKs) are important in the transmission of biochemical signals across the plasma membrane of cells. These transmembrane molecules characteristically consist of an extracellular ligand-binding domain connected through a segment in the plasma membrane to an intracellular tyrosine kinase domain. Binding of ligand to the receptor results in stimulation of the receptor-associated tyrosine kinase activity which leads to phosphorylation of tyrosine residues on both the receptor and other intracellular molecules. These changes in tyrosine phosphorylation initiate a signalling cascade leading to a variety of cellular responses. To date, at least nineteen distinct RTK subfamilies, defined by amino acid sequence homology, have been identified. One of these subfamilies is presently comprised by the fms-like tyrosine kinase receptor, Flt-1 (also referred to as VEGFR-1), the kinase insert domain-containing receptor, KDR (also referred to as VEGFR-2 or Flk-1), and another fms-like tyrosine kinase receptor, Flt-4. Two of these related RTKs, Flt-1 and KDR, have been shown to bind VEGF with high affinity (De Vries et al, 1992, Science 255: 989-991; Terman et al, 1992, Biochem. Biophys. Res. Comm. 1992, 187: 1579-1586). Binding of VEGF to these receptors expressed in heterologous cells has been associated with changes in the tyrosine phosphorylation status of cellular proteins and calcium fluxes.

VEGF is a key stimulus for vasculogenesis and angiogenesis. This cytokine induces a vascular sprouting phenotype by inducing endothelial cell proliferation, protease expression and migration, and subsequent organisation of cells to form a capillary tube (Keck, P. J., Hauser, S. D., Krivi, G., Sanzo, K., Warren, T., Feder, J., and Connolly, D. T., Science (Washington D.C.), 246: 1309-1312, 1989; Lamoreaux, W. J., Fitzgerald, M. E., Reiner, A., Hasty, K. A., and Charles, S. T., Microvasc. Res., 55: 29-42, 1998; Pepper, M. S., Montesano, R., Mandroita, S. J., Orci, L. and Vassalli, J. D., Enzyme Protein, 49:138-162, 1996.). In addition, VEGF induces significant vascular permeability (Dvorak, H. F., Detmar, M., Claffey, K. P., Nagy, J. A., van de Water, L., and Senger, D. R., (Int. Arch. Allergy Immunol., 107: 233-235, 1995; Bates, D. O., Heald, R. I., Curry, F. E. and Williams, B. J. Physiol. (Lond.), 533: 263-272, 2001), promoting formation of a hyper-permeable, immature vascular network which is characteristic of pathological angiogenesis.

It has been shown that activation of KDR alone is sufficient to promote all of the major phenotypic responses to VEGF, including endothelial cell proliferation, migration, and survival, and the induction of vascular permeability (Meyer, M., Clauss, M., Lepple-Wienhues, A., Waltenberger, J., Augustin, H. G., Ziche, M., Lanz, C., Büttner, M., Rziha, H-J., and Dehio, C., EMBO J., 18: 363-374, 1999; Zeng, H., Sanyal, S. and Mukhopadhyay, D., J. Biol. Chem., 276: 32714-32719, 2001; Gille, H., Kowalski, J., Li, B., LeCouter, J., Moffat, B, Zioncheck, T. F., Pelletier, N. and Ferrara, N., J. Biol. Chem., 276: 3222-3230, 2001).

Quinazoline derivatives which are inhibitors of VEGF receptor tyrosine kinase are described in International Patent Applications Publication Nos. WO 98/13354 and WO 01/32651. In WO 98/13354 and WO 01/32651 compounds are described which possess activity against VEGF receptor tyrosine kinase (VEGF RTK) whilst possessing some activity against epidermal growth factor (EGF) receptor tyrosine kinase (EGF RTK). ZD6474 is 4-(4-bromo-2-fluoroanilino)-6-methoxy-7-(1-methylpiperidin-4-ylmethoxy)quinazoline:

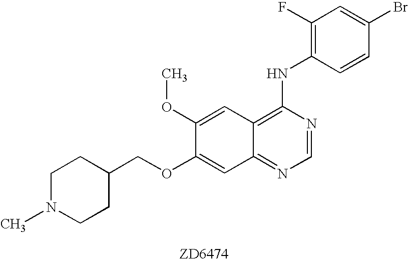

ZD6474 falls within the broad general disclosure of WO 98/13354 and is exemplified in WO 01/32651. ZD6474 is a potent inhibitor of VEGF RTK and also has some activity against EGF RTK. ZD6474 has been shown to elicit broad-spectrum anti-tumour activity in a range of models following once-daily oral administration (Wedge S. R., Ogilvie D. J., Dukes M. et al, Proc. Am. Assoc. Canc. Res. 2001; 42: abstract 3126).

In WO 98/13354 and WO 01/32651 it is stated that compounds of their inventions: “may be applied as a sole therapy or may involve, in addition to a compound of the invention, one or more other substances and/or treatments. Such conjoint treatment may be achieved by way of the simultaneous, sequential or separate administration of the individual components of the treatment.”

WO 98/13354 and WO 01/32651 then go on to describe examples of such conjoint treatment including surgery, radiotherapy and various types of chemotherapeutic agent including “inhibitors of growth factor function, (such growth factors include for example platelet derived growth factor and hepatocyte growth factor such inhibitors include growth factor antibodies, growth factor receptor antibodies, tyrosine kinase inhibitors and serine/threonine kinase inhibitors)”.

Nowhere in WO 98/13354 and WO 01/32651 is the specific combination of ZD6474 and imatinib suggested.

Nowhere in WO 98/13354 and WO 01/32651 does it state that use of any compound of the invention therein with other treatments will produce surprisingly beneficial effects.

Imatinib (also known as Glivec® or Gleevec®) is a protein tyrosine kinase inhibitor that inhibits Bcr-Abl tyrosine kinase. Imatinib also inhibits platelet derived growth factor receptor tyrosine kinase (PDGF RTK) and stem cell factor receptor tyrosine kinase (SCF RTK, c-kit). It is known that imatinib only targets mutated c-kit. Imatinib has been used, in particular, in the treatment of chronic myelogenous leukaemia (CML) and in the treatment of gastrointestinal stromal tumours (GIST).

Imatinib may also be effective in myeloproliferative disorders for example chronic eosinophilic leukaemia, hypereosinophilic syndrome and polycythaemia rubra vera (Apperley J F et al New Engl J Med. 2002; 347:481-487 and Silver R T et al Blood, 2004; 104:11. Abstract 656) and also in myelodysplastic syndrome for example chronic myelomonocytic leukaemia (CMML) and myelofibrosis with myeloid metaplasia (Blood. 2004 Oct. 1; 104(7):1931-9. Epub 2004 May 27).

c-Kit and its ligand SCF have been found in numerous solid and haematological malignancies, including gastrointestinal stromal tumours, primary brain tumours such as glioblastoma, glioma and medulloblastoma, small cell lung cancer (SCLC), malignant mesothelioma, tumours of the testis such as seminoma and testicular teratocarcinoma, tumours of the ovary such as dysgerminoma and gonadoblastoma, chronic myelogenous leukaemia (CML), acute myelogenous leukaemia (AML) and mastocytosis (see for example Jnl. Clin. Oncol., 2004, 22, 4514-4522). c-Kit has also been found in hepatocellular carcinoma, (Am J Clin Pathol. 2005 July; 124(1):31-6), and colorectal carcinoma, (Case Reports Tumour Biol. 1993; 14(5):295-302). c-Kit is an important signal transduction inhibitor in certain cancers such as gastrointestinal tumours (GIST), (Bümming et al, 2003 Br J Cancer 89, 460-464), small cell lung cancer (SCLC), (Pott et. al., 2003, Annals of Oncology 14: 894-879), and chronic myelogenous leukaemia (CML), (Goselink et al. 1992, Blood 80, 750-757 and Muroi et al, 1995, Leuk Lymphoma 16, 297-305). c-Kit is also an important signal transduction inhibitor in soft tissue sarcomas like leiomyosarcoma.

Unexpectedly and surprisingly we have now found that the particular compound ZD6474 used in combination with a particular selection from the broad description of combination therapies listed in WO 98/13354 and WO 01/32651, namely with imatinib, produces significantly better effects than any one of ZD6474 and imatinib used alone. In particular, ZD6474 used in combination with imatinib produces significantly better effects on solid tumours than any one of ZD6474 and imatinib used alone.

Anti-cancer effects of a method of treatment of the present invention include, but are not limited to, anti-tumour effects, the response rate, the time to disease progression and the survival rate. Anti-tumour effects of a method of treatment of the present invention include but are not limited to, inhibition of tumour growth, tumour growth delay, regression of tumour, shrinkage of tumour, increased time to regrowth of tumour on cessation of treatment, slowing of disease progression. It is expected that when a method of treatment of the present invention is administered to a warm-blooded animal such as a human, in need of treatment for cancer, with or without a solid tumour, said method of treatment will produce an effect, as measured by, for example, one or more of: the extent of the anti-tumour effect, the response rate, the time to disease progression and the survival rate. Anti-cancer effects include prophylactic treatment as well as treatment of existing disease.

According to the present invention there is provided a method for the production of an antiangiogenic and/or vascular permeability reducing effect in a warm-blooded animal such as a human, which comprises administering to said animal an effective amount of ZD6474 or a pharmaceutically acceptable salt thereof, before, after or simultaneously with an effective amount of imatinib.

According to a further aspect of the present invention there is provided a method for the treatment of a cancer in a warm-blooded animal such as a human, which comprises administering to said animal an effective amount of ZD6474 or a pharmaceutically acceptable salt thereof, before, after or simultaneously with an effective amount of imatinib.

According to a further aspect of the present invention there is provided a method for the treatment of a cancer involving a solid tumour in a warm-blooded animal such as a human, which comprises administering to said animal an effective amount of ZD6474 or a pharmaceutically acceptable salt thereof, before, after or simultaneously with an effective amount of imatinib.

According to a further aspect of the present invention there is provided a method for the treatment of a gastrointestinal stromal tumour (GIST) in a warm-blooded animal such as a human, which comprises administering to said animal an effective amount of ZD6474 or a pharmaceutically acceptable salt thereof, before, after or simultaneously with an effective amount of imatinib.

According to a further aspect of the present invention there is provided a method for the treatment of a leukaemia in a warm-blooded animal such as a human, which comprises administering to said animal an effective amount of ZD6474 or a pharmaceutically acceptable salt thereof, before, after or simultaneously with an effective amount of imatinib.

According to a further aspect of the present invention there is provided a method for the treatment of chronic myelogenous leukaemia (CML) in a warm-blooded animal such as a human, which comprises administering to said animal an effective amount of ZD6474 or a pharmaceutically acceptable salt thereof, before, after or simultaneously with an effective amount of imatinib.

According to a further aspect of the present invention there is provided a method for the treatment of small cell lung cancer (SCLC) in a warm-blooded animal such as a human, which comprises administering to said animal an effective amount of ZD6474 or a pharmaceutically acceptable salt thereof, before, after or simultaneously with an effective amount of imatinib.

According to a further aspect of the present invention there is provided a method for the production of an antiangiogenic and/or vascular permeability reducing effect in a warm-blooded animal such as a human, which comprises administering to said animal an effective amount of ZD6474 or a pharmaceutically acceptable salt thereof, before, after or simultaneously with an effective amount of imatinib; wherein ZD6474 and imatinib may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of ZD6474 or a pharmaceutically acceptable salt thereof, before, after or simultaneously with an effective amount of imatinib; wherein ZD6474 and imatinib may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of ZD6474 or a pharmaceutically acceptable salt thereof, before, after or simultaneously with an effective amount of imatinib; wherein ZD6474 and imatinib may each optionally be administered together with a pharmaceutically acceptable excipient or carrier.

According to a further aspect of the present invention there is provided a method for the treatment of a gastrointestinal stromal tumour (GIST) in a warm-blooded animal such as a human, which comprises administering to said animal an effective amount of ZD6474 or a pharmaceutically acceptable salt thereof, before, after or simultaneously with an effective amount of imatinib; wherein ZD6474 and imatinib may each optionally be administered together with a pharmaceutically acceptable excipient or carrier.

According to a further aspect of the present invention there is provided a method for the treatment of a leukaemia in a warm-blooded animal such as a human, which comprises administering to said animal an effective amount of ZD6474 or a pharmaceutically acceptable salt thereof, before, after or simultaneously with an effective amount of imatinib; wherein ZD6474 and imatinib may each optionally be administered together with a pharmaceutically acceptable excipient or carrier.

According to a further aspect of the present invention there is provided a method for the treatment of chronic myelogenous leukaemia (CML) in a warm-blooded animal such as a human, which comprises administering to said animal an effective amount of ZD6474 or a pharmaceutically acceptable salt thereof, before, after or simultaneously with an effective amount of imatinib; wherein ZD6474 and imatinib may each optionally be administered together with a pharmaceutically acceptable excipient or carrier.

According to a further aspect of the present invention there is provided a method for the treatment of small cell lung cancer (SCLC) in a warm-blooded animal such as a human, which comprises administering to said animal an effective amount of ZD6474 or a pharmaceutically acceptable salt thereof, before, after or simultaneously with an effective amount of imatinib; wherein ZD6474 and imatinib may each optionally be administered together with a pharmaceutically acceptable excipient or carrier.

According to a further aspect of the invention there is provided a pharmaceutical composition which comprises ZD6474 or a pharmaceutically acceptable salt thereof, and imatinib, in association with a pharmaceutically acceptable excipient or carrier.

According to a further aspect of the present invention there is provided a combination product comprising ZD6474 or a pharmaceutically acceptable salt thereof, and imatinib, for use in a method of treatment of a human or animal body by therapy.

According to a further aspect of the present invention there is provided a kit comprising ZD6474 or a pharmaceutically acceptable salt thereof, and imatinib.

According to a further aspect of the present invention there is provided a kit comprising:

a) ZD6474 or a pharmaceutically acceptable salt thereof in a first unit dosage form;
b) imatinib in a second unit dosage form; and
c) container means for containing said first and second dosage forms.

According to a further aspect of the present invention there is provided a kit comprising:

a) ZD6474 or a pharmaceutically acceptable salt thereof, together with a pharmaceutically acceptable excipient or carrier, in a first unit dosage form;
b) imatinib together with a pharmaceutically acceptable excipient or carrier, in a second unit dosage form; and
c) container means for containing said first and second dosage forms.

According to a further aspect of the present invention there is provided the use of ZD6474 or a pharmaceutically acceptable salt thereof and imatinib in the manufacture of a medicament for use in the production of an antiangiogenic and/or vascular permeability reducing effect in a warm-blooded animal such as a human.

According to a further aspect of the present invention there is provided the use of ZD6474 or a pharmaceutically acceptable salt thereof and imatinib in the manufacture of a medicament for use in the production of an anti-cancer effect in a warm-blooded animal such as a human.

According to a further aspect of the present invention there is provided the use of ZD6474 or a pharmaceutically acceptable salt thereof and imatinib in the manufacture of a medicament for use in the production of an anti-tumour effect in a warm-blooded animal such as a human.

According to a further aspect of the present invention there is provided the use of ZD6474 or a pharmaceutically acceptable salt thereof and imatinib in the manufacture of a medicament for use in the production of an anti-tumour effect in a warm-blooded animal such as a human wherein the tumour is a gastrointestinal stromal tumour (GIST).

According to a further aspect of the present invention there is provided the use of ZD6474 or a pharmaceutically acceptable salt thereof and imatinib in the manufacture of a medicament for use in the production of an anti-cancer effect in a warm-blooded animal such as a human wherein the cancer is a leukaemia.

According to a further aspect of the present invention there is provided the use of ZD6474 or a pharmaceutically acceptable salt thereof and imatinib in the manufacture of a medicament for use in the production of an anti-cancer effect in a warm-blooded animal such as a human wherein the cancer is chronic myelogenous leukaemia (CML).

According to a further aspect of the present invention there is provided the use of ZD6474 or a pharmaceutically acceptable salt thereof and imatinib in the manufacture of a medicament for use in the production of an anti-cancer effect in a warm-blooded animal such as a human wherein the cancer is small cell lung cancer (SCLC).

According to a further aspect of the present invention there is provided a therapeutic combination treatment comprising the administration of an effective amount of ZD6474 or a pharmaceutically acceptable salt thereof, optionally together with a pharmaceutically acceptable excipient or carrier, and the simultaneous, sequential or separate administration of an effective amount of imatinib, wherein imatinib may optionally be administered together with a pharmaceutically acceptable excipient or carrier, to a warm-blooded animal such as a human in need of such therapeutic treatment. Such therapeutic treatment includes an antiangiogenic and/or vascular permeability effect, an anti-cancer effect and an anti-tumour effect.

A combination treatment of the present invention as defined herein may be achieved by way of the simultaneous, sequential or separate administration of the individual components of said treatment. A combination treatment as defined herein may be applied as a sole therapy or may involve surgery or radiotherapy or an additional chemotherapeutic agent in addition to a combination treatment of the invention.

Surgery may comprise the step of partial or complete tumour resection, prior to, during or after the administration of the combination treatment with ZD6474 described herein.

Other chemotherapeutic agents for optional use with a combination treatment of the present invention include those described in WO 01/32651 which is incorporated herein by reference. Such chemotherapy may cover five main categories of therapeutic agent:

(i) other antiangiogenic agents including vascular targeting agents;

(ii) cytostatic agents;

(iii) biological response modifiers (for example interferon);

(iv) antibodies (for example edrecolomab); and

(v) antiproliferative/antineoplastic drugs and combinations thereof, as used in medical oncology; and other categories of agent are:

(vi) antisense therapies;

(vii) gene therapy approaches; and

(ix) immunotherapy approaches.

Particular examples of chemotherapeutic agents for use with a combination treatment of the present invention are raltitrexed, etoposide, vinorelbine, paclitaxel, docetaxel, cisplatin, oxaliplatin, carboplatin, gemcitabine, irinotecan (CPT-11), 5-fluorouracil (5-FU, (including capecitabine)) and hydroxyurea. Such combinations are expected to be particularly useful for the treatment of cancer of the lung, head and neck, brain, colon, rectum, oesophagus, stomach, cervix, ovary, skin, breast, bladder, prostate, pancreas, liver, thyroid and including haematological malignancies. Such combinations are expected to be more particularly useful for the treatment of gastrointestinal stromal tumours (GIST), small cell lung cancer (SCLC), glioblastoma multiforme (GBM), malignant glioma, malignant mesothelioma, mastocytosis and leukaemias such as acute myelogenous leukaemia (AML) and chronic myelogenous leukaemia (CML). Such combinations are expected to be especially useful for the treatment of gastrointestinal stromal tumours (GIST), small cell lung cancer (SCLC), and leukaemias such as chronic myelogenous leukaemia (CML). Such combinations are also expected to be particularly useful for the treatment of hepatocellular carcinoma (HCC). Such combinations are also expected to be particularly useful for the treatment of thyroid cancer. Such combinations are also expected to be particularly useful for the treatment of soft tissue sarcomas. Such combinations are also expected to be particularly useful for the treatment of myeloproliferative disorders and myelodysplastic syndrome.

The administration of a triple combination of ZD6474, imatinib and ionising radiation may produce effects, such as anti-tumour effects, greater than those achieved with any of ZD6474, imatinib and ionising radiation used alone, greater than those achieved with the combination of ZD6474 and imatinib, greater than those achieved with the combination of ZD6474 and ionising radiation, greater than those achieved with the combination of imatinib and ionising radiation.

According to the present invention there is provided a method for the production of an antiangiogenic and/or vascular permeability reducing effect in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation.

According to a further aspect of the present invention there is provided a method for the treatment of a cancer in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation.

According to a further aspect of the present invention there is provided a method for the treatment of a cancer involving a solid tumour in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation.

According to a further aspect of the present invention there is provided a method for the treatment of a gastrointestinal stromal tumour (GIST) in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation.

According to a further aspect of the present invention there is provided a method for the treatment of a leukaemia in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation.

According to a further aspect of the present invention there is provided a method for the treatment of chronic myelogenous leukaemia (CML) in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation.

According to a further aspect of the present invention there is provided a method for the treatment of small cell lung cancer (SCLC) in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation.

According to a further aspect of the present invention there is provided a method for the production of an antiangiogenic and/or vascular permeability reducing effect in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation, wherein ZD6474 and imatinib may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation, wherein ZD6474 and imatinib may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation, wherein ZD6474 and imatinib may each optionally be administered together with a pharmaceutically acceptable excipient or carrier.

According to a further aspect of the present invention there is provided a method for the treatment of a gastrointestinal stromal tumour (GIST) in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation, wherein ZD6474 and imatinib may each optionally be administered together with a pharmaceutically acceptable excipient or carrier.

According to a further aspect of the present invention there is provided a method for the treatment of a leukaemia in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation, wherein ZD6474 and imatinib may each optionally be administered together with a pharmaceutically acceptable excipient or carrier.

According to a further aspect of the present invention there is provided a method for the treatment of chronic myelogenous leukaemia (CML) in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation, wherein ZD6474 and imatinib may each optionally be administered together with a pharmaceutically acceptable excipient or carrier.

According to a further aspect of the present invention there is provided a method for the treatment of small cell lung cancer (SCLC) in a warm-blooded animal such as a human, which comprises administering to said animal an effective amount of ZD6474 or a pharmaceutically acceptable salt thereof, before, after or simultaneously with an effective amount of imatinib and before, after or simultaneously with an effective amount of ionising radiation, wherein ZD6474 and imatinib may each optionally be administered together with a pharmaceutically acceptable excipient or carrier.

According to a further aspect of the present invention there is provided the use of ZD6474 or a pharmaceutically acceptable salt thereof and imatinib in the manufacture of a medicament for use in the production of an antiangiogenic and/or vascular permeability reducing effect in a warm-blooded animal such as a human which is being treated with ionising radiation.

According to a further aspect of the present invention there is provided the use of ZD6474 or a pharmaceutically acceptable salt thereof and imatinib in the manufacture of a medicament for use in the production of an anti-cancer effect in a warm-blooded animal such as a human which is being treated with ionising radiation.

According to a further aspect of the present invention there is provided the use of ZD6474 or a pharmaceutically acceptable salt thereof and imatinib in the manufacture of a medicament for use in the production of an anti-tumour effect in a warm-blooded animal such as a human which is being treated with ionising radiation.

According to a further aspect of the present invention there is provided the use of ZD6474 or a pharmaceutically acceptable salt thereof and imatinib in the manufacture of a medicament for use in the production of an anti-tumour effect in a warm-blooded animal such as a human which is being treated with ionising radiation wherein the tumour is a gastrointestinal stromal tumour (GIST).

According to a further aspect of the present invention there is provided the use of ZD6474 or a pharmaceutically acceptable salt thereof and imatinib in the manufacture of a medicament for use in the production of an anti-cancer effect in a warm-blooded animal such as a human which is being treated with ionising radiation wherein the cancer is a leukaemia.

According to a further aspect of the present invention there is provided the use of ZD6474 or a pharmaceutically acceptable salt thereof and imatinib in the manufacture of a medicament for use in the production of an anti-cancer effect in a warm-blooded animal such as a human which is being treated with ionising radiation wherein the cancer is chronic myelogenous leukaemia (CML).

According to a further aspect of the present invention there is provided the use of ZD6474 or a pharmaceutically acceptable salt thereof and imatinib in the manufacture of a medicament for use in the production of an anti-cancer effect in a warm-blooded animal such as a human which is being treated with ionising radiation wherein the cancer is small cell lung cancer (SCLC).

According to a further aspect of the present invention there is provided a therapeutic combination treatment comprising the administration of an effective amount of ZD6474 or a pharmaceutically acceptable salt thereof, optionally together with a pharmaceutically acceptable excipient or carrier, and the administration of an effective amount of imatinib, optionally together with a pharmaceutically acceptable excipient or carrier and the administration of an effective amount of ionising radiation, to a warm-blooded animal such as a human in need of such therapeutic treatment wherein the ZD6474, imatinib and ionising radiation may be administered simultaneously, sequentially or separately and in any order.

A warm-blooded animal such as a human which is being treated with ionising radiation means a warm-blooded animal such as a human which is treated with ionising radiation before, after or at the same time as the administration of a medicament or combination treatment comprising ZD6474 and imatinib. For example said ionising radiation may be given to said warm-blooded animal such as a human within the period of a week before to a week after the administration of a medicament or combination treatment comprising ZD6474 and imatinib. This means that ZD6474, imatinib and ionising radiation may be administered separately or sequentially in any order, or may be administered simultaneously. The warm-blooded animal may experience the effect of each of ZD6474, imatinib and radiation simultaneously.

According to one aspect of the present invention the ionising radiation is administered before one of ZD6474 and imatinib or after one of ZD6474 and imatinib.

According to one aspect of the present invention the ionising radiation is administered before both ZD6474 and imatinib or after both ZD6474 and imatinib.

According to one aspect of the present invention ZD6474 is administered to a warm-blooded animal after the animal has been treated with ionising radiation. According to another aspect of the present invention the effect of a method of treatment of the present invention is expected to be at least equivalent to the addition of the effects of each of the components of said treatment used alone, that is, of each of ZD6474 and imatinib used alone or of each of ZD6474, imatinib and ionising radiation used alone.

According to another aspect of the present invention the effect of a method of treatment of the present invention is expected to be greater than the addition of the effects of each of the components of said treatment used alone, that is, of each of ZD6474 and imatinib used alone or of each of ZD6474, imatinib and ionising radiation used alone.

According to another aspect of the present invention the effect of a method of treatment of the present invention is expected to be a synergistic effect.

According to the present invention a combination treatment is defined as affording a synergistic effect if the effect is therapeutically superior, as measured by, for example, the extent of the response, the response rate, the time to disease progression or the survival period, to that achievable on dosing one or other of the components of the combination treatment at its conventional dose. For example, the effect of the combination treatment is synergistic if the effect is therapeutically superior to the effect achievable with ZD6474 or imatinib or ionising radiation alone. Further, the effect of the combination treatment is synergistic if a beneficial effect is obtained in a group of patients that does not respond (or responds poorly) to ZD6474 or imatinib or ionising radiation alone. In addition, the effect of the combination treatment is defined as affording a synergistic effect if one of the components is dosed at its conventional dose and the other component(s) is/are dosed at a reduced dose and the therapeutic effect, as measured by, for example, the extent of the response, the response rate, the time to disease progression or the survival period, is equivalent to that achievable on dosing conventional amounts of the components of the combination treatment. In particular, synergy is deemed to be present if the conventional dose of ZD6474 or imatinib or ionising radiation may be reduced without detriment to one or more of the extent of the response, the response rate, the time to disease progression and survival data, in particular without detriment to the duration of the response, but with fewer and/or less troublesome side-effects than those that occur when conventional doses of each component are used.

As stated above the combination treatments of the present invention as defined herein are of interest for their antiangiogenic and/or vascular permeability effects. Angiogenesis and/or an increase in vascular permeability is present in a wide range of disease states including cancer (including leukaemia, multiple myeloma and lymphoma), diabetes, psoriasis, rheumatoid arthritis, Kaposi's sarcoma, haemangioma, acute and chronic nephropathies, atheroma, arterial restenosis, autoimmune diseases, acute inflammation, lymphoedema, endometriosis, dysfunctional uterine bleeding and ocular diseases with retinal vessel proliferation including age-related macular degeneration. Combination treatments of the present invention are expected to be particularly useful in the prophylaxis and treatment of diseases such as cancer and Kaposi's sarcoma. In particular such combination treatments of the invention are expected to slow advantageously the growth of primary and recurrent solid tumours of, for example, the colon, pancreas, brain, bladder, breast, prostate, thyroid, lungs and skin. Combination treatments of the present invention are expected to slow advantageously the growth of tumours in colorectal cancer and in lung cancer, for example mesothelioma, small cell lung cancer (SCLC) and non-small cell lung cancer (NSCLC). More particularly such combination treatments of the invention are expected to inhibit any form of cancer associated with VEGF including leukaemia, multiple myeloma and lymphoma and also, for example, to inhibit the growth of those primary and recurrent solid tumours which are associated with VEGF, especially those tumours which are significantly dependent on VEGF for their growth and spread, including for example, certain tumours of the colon (including rectum), pancreas, brain, bladder, breast, prostate, lung, vulva, skin and particularly NSCLC. More especially combination treatments of the present invention are expected to slow advantageously the growth of gastrointestinal stromal tumours (GIST). More especially combination treatments of the present invention are expected to slow advantageously the growth of tumours in small cell lung cancer (SCLC). More especially combination treatments of the present invention are expected to slow advantageously the growth of hepatocellular carcinoma (HCC). More especially combination treatments of the present invention are expected to slow advantageously the growth of soft tissue sarcomas such as leiomyosarcoma. More especially combination treatments of the present invention are expected to inhibit leukaemias particularly chronic myelogenous leukaemia (CML). In particular combination treatments of the present invention are expected to inhibit myeloproliferative disorders and myelodysplastic syndrome. In particular combination treatments of the present invention are expected to slow advantageously the growth of tumours of the brain such as malignant glioma and glioblastoma multiforme (GBM). In particular combination treatments of the present invention are expected to slow advantageously the growth of tumours of the thyroid.

In another aspect of the present invention ZD6474 and imatinib, optionally with ionising radiation, are expected to inhibit the growth of those primary and recurrent solid tumours which are associated with VEGF especially those tumours which are significantly dependent on VEGF for their growth and spread.

In another aspect of the present invention ZD6474 and imatinib, optionally with ionising radiation, are expected to inhibit the growth of those primary and recurrent solid tumours which are associated with both VEGF and EGF especially those tumours which are significantly dependent on VEGF and EGF for their growth and spread.

The compositions described herein may be in a form suitable for oral administration, for example as a tablet or capsule, for nasal administration or administration by inhalation, for example as a powder or solution, for parenteral injection (including intravenous, subcutaneous, intramuscular, intravascular or infusion) for example as a sterile solution, suspension or emulsion, for topical administration for example as an ointment or cream, for rectal administration for example as a suppository or the route of administration may be by direct injection into the tumour or by regional delivery or by local delivery. In other embodiments of the present invention the ZD6474 of the combination treatment may be delivered endoscopically, intratracheally, intralesionally, percutaneously, intravenously, subcutaneously, intraperitoneally or intratumourally. Preferably ZD6474 is administered orally. In general the compositions described herein may be prepared in a conventional manner using conventional excipients. The compositions of the present invention are advantageously presented in unit dosage form.

ZD6474 will normally be administered to a warm-blooded animal at a unit dose within the range 10-500 mg per square metre body area of the animal, for example approximately 0.3-15 mg/kg in a human. A unit dose in the range, for example, 0.3-15 mg/kg, preferably 0.5-5 mg/kg is envisaged and this is normally a therapeutically-effective dose. A unit dosage form such as a tablet or capsule will usually contain, for example 25-500 mg of active ingredient. Preferably a daily dose in the range of 0.5-5 mg/kg is employed.

Imatinib may be dosed according to known routes of administration and dosages.

For example imatinib may be dosed at 400 mg/day for patients in chronic phase CML.

For example imatinib may be dosed at 400-800 mg/day for patients in accelerated phase CML.

For example imatinib may be dosed at 600 mg/day for patients in blast crisis CML.

For example imatinib may be dosed at 400 mg-600 mg/day for patients with GIST.

The dosages and schedules may vary according to the particular disease state and the overall condition of the patient. Dosages and schedules may also vary if, in addition to a combination treatment of the present invention, one or more additional chemotherapeutic agents is/are used. Scheduling can be determined by the practitioner who is treating any particular patient.

Radiotherapy may be administered according to the known practices in clinical radiotherapy. The dosages of ionising radiation will be those known for use in clinical radiotherapy. The radiation therapy used will include for example the use of γ-rays, X-rays, and/or the directed delivery of radiation from radioisotopes. Other forms of DNA damaging factors are also included in the present invention such as microwaves and UV-irradiation. For example X-rays may be dosed in daily doses of 1.8-2.0Gy, 5 days a week for 5-6 weeks. Normally a total fractionated dose will lie in the range 45-60Gy. Single larger doses, for example 5-10Gy may be administered as part of a course of radiotherapy. Single doses may be administered intraoperatively. Hyperfractionated radiotherapy may be used whereby small doses of X-rays are administered regularly over a period of time, for example 0.1Gy per hour over a number of days. Dosage ranges for radioisotopes vary widely, and depend on the half-life of the isotope, the strength and type of radiation emitted, and on the uptake by cells.

The size of the dose of each therapy which is required for the therapeutic or prophylactic treatment of a particular disease state will necessarily be varied depending on the host treated, the route of administration and the severity of the illness being treated. Accordingly the optimum dosage may be determined by the practitioner who is treating any particular patient. For example, it may be necessary or desirable to reduce the above-mentioned doses of the components of the combination treatments in order to reduce toxicity.

The present invention relates to combinations of imatinib with ZD6474 or with a salt of ZD6474.

Salts of ZD6474 for use in pharmaceutical compositions will be pharmaceutically acceptable salts, but other salts may be useful in the production of ZD6474 and its pharmaceutically acceptable salts. Such salts may be formed with an inorganic or organic base which affords a pharmaceutically acceptable cation. Such salts with inorganic or organic bases include for example an alkali metal salt, such as a sodium or potassium salt, an alkaline earth metal salt such as a calcium or magnesium salt, an ammonium salt or for example a salt with methylamine, dimethylamine, trimethylamine, piperidine, morpholine or tris-(2-hydroxyethyl)amine.

ZD6474 may be synthesised according to any of the known processes for making ZD6474. For example ZD6474 may be made according to any of the processes described in WO 01/32651; for example those described in Examples 2(a), 2(b) and 2(c) of WO 01/32651.

Imatinib is commercially available.

### The following test may be used to demonstrate the activity of ZD6474 in combination with imatinib.

### C6 Rat Glial Xenograft Model

Tumour implantation procedures were performed on mice of at least 8 weeks of age. Rat tumour xenografts were grown in female athymic (nu/nu genotype, Swiss) mice. C6 Rat glial cells (1×10⁴ per mouse) were injected subcutaneously (s.c.) in the right flanks of the experimental athymic mice. Ten days after cellular implant, tumours were established and mice randomised into groups (10 animals/group) before treatment was started. ZD6474 and imatinib were each suspended in a 1% (v/v) aqueous solution of polyoxyethylene (20) sorbitan mono-oleate and administered by once daily oral gavage. When ZD6474 and imatinib were given in combination they were co-formulated in a single solution before being administered. Solutions were dosed at a volume of 10 ml/kg body weight. Animals were treated with either imatinib (150 mg/kg/day) alone, ZD6474 (12.5 mg/kg/day) alone, or imatinib (150 mg/kg/day) and ZD6474 (12.5 mg/kg/day) for the duration of the study. Tumour volumes were assessed from the start of treatment by bilateral Vernier caliper measurement. Growth inhibition from the start of treatment was assessed by comparison of the differences in tumour volume between control and treated groups.

Statistical significance was evaluated using a one-tailed t-test.

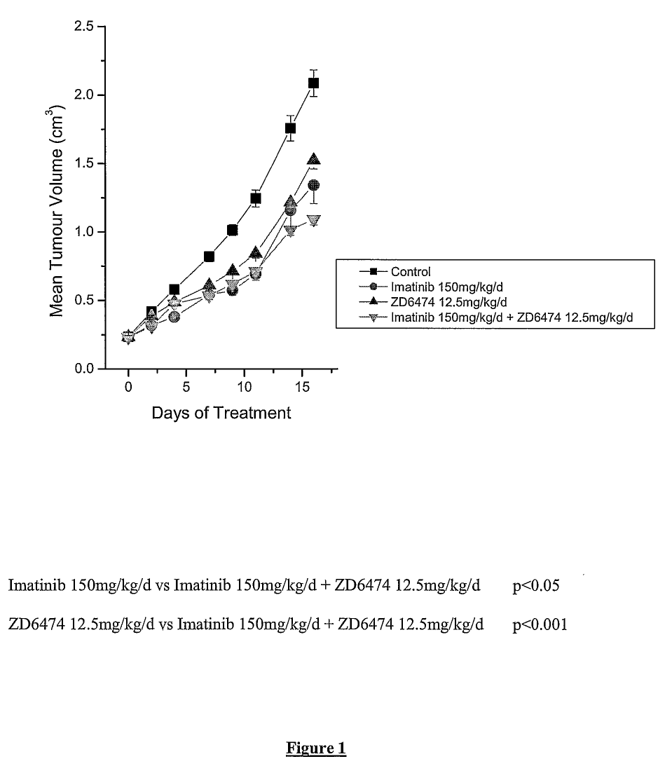

The data is shown in FIG. 1. The growth inhibition of tumours was significantly greater with the combination of the two agents, ZD6474 (12.5 mg/kg/day) and imatinib (150 mg/kg/day), than with either agent alone.

An analogous experiment may be used to look at the combination of ZD6474 and imatinib with ionising radiation.

1-10. (canceled)

11. A pharmaceutical composition which comprises ZD6474 or a pharmaceutically acceptable salt thereof, and imatinib, in association with a pharmaceutically acceptable excipient or carrier.

12. A kit comprising ZD6474 or a pharmaceutically acceptable salt thereof, and imatinib.

13. A method for the production of an antiangiogenic and/or vascular permeability reducing effect in a warm-blooded animal, which comprises administering to said animal an effective amount of ZD6474 or a pharmaceutically acceptable salt thereof, before, after or simultaneously with an effective amount of imatinib.

14. A method for the production of an antiangiogenic and/or vascular permeability reducing effect in a warm-blooded animal, which comprises administering to said animal an effective amount of ZD6474 or a pharmaceutically acceptable salt thereof, before, after or simultaneously with an effective amount of imatinib and before, after or simultaneously with an effective amount of ionising radiation.

15. The method according to claim 13 wherein said method produces an anti-cancer effect in a warm-blooded animal, which method comprises administering to said animal an effective amount of ZD6474 or a pharmaceutically acceptable salt thereof, before, after or simultaneously with an effective amount of imatinib.

16. The method according to claim 14 wherein said method produces an anti-cancer effect in a warm-blooded animal, which method comprises administering to said animal an effective amount of ZD6474 or a pharmaceutically acceptable salt thereof, before, after or simultaneously with an effective amount of imatinib and before, after or simultaneously with an effective amount of ionising radiation.

17. The method according to claim 13 wherein said method produces an anti-tumour effect in a warm-blooded animal, which method comprises administering to said animal an effective amount of ZD6474 or a pharmaceutically acceptable salt thereof, before, after or simultaneously with an effective amount of imatinib.

18. A method according to claim 14 for the production of an anti-tumour effect in a warm-blooded animal, which method comprises administering to said animal an effective amount of ZD6474 or a pharmaceutically acceptable salt thereof, before, after or simultaneously with an effective amount of imatinib and before, after or simultaneously with an effective amount of ionising radiation.

19. The method of claim 17 or claim 18 for the treatment of a gastrointestinal stromal tumour (GIST).

20. The method of claim 15 or claim 16 for the treatment of a small cell lung cancer (SCLC).

21. The method of claim 15 or claim 16 for the treatment of a leukaemia.

22. The method of claim 15 or claim 16 wherein the cancer is chronic myelogenous leukaemia CML).
